(19) United States
(12) Patent Application Publication
(10) Pub. No.: US 2023/0013964 A1
(43) Pub. Date: Jan. 19, 2023

(54) Rare Sugars in Food and Beverage Products

(71) Applicant: Ocean Spray Cranberries, Inc., Lakeville-Middleboro, MA (US)

(72) Inventors: Kristen Girard, Lakeville-Middleboro, MA (US); Meredith Myers, Lakeville-Middleboro, MA (US)

(21) Appl. No.: 17/842,647

(22) Filed: Jun. 16, 2022

Related U.S. Application Data
(60) Provisional application No. 63/211,991, filed on Jun. 17, 2021.

(51) Int. Cl. A23L 27/30; A23L 2/60; A23L 19/00; A23L 2/08

(52) U.S. Cl. CPC A23L 27/33; A23L 2/60; A23L 19/05; A23L 2/08; A23V 2002/00

(57) ABSTRACT

This disclosure relates to a product, a beverage product, and an infusion formulation that each include at least two rare sugars, for example allulose and tagatose. The combination of allulose and tagatose synergistically sweeten the product, beverage product, and infusion formulation. The product includes a food body having a food component and an infusion component. The infusion component includes at least two rare sugars. The beverage product includes a consumable liquid and at least two rare sugars. This disclosure further relates to methods for preparing a food product and beverage product.

### CROSS REFERENCE TO RELATED APPLICATIONS

The present application claims priority to U.S. Provisional Application No. 63/211,991, filed in on Jun. 17, 2021, the disclosures of which are expressly incorporated herein in entirety by reference thereto.

### TECHNICAL FIELD

This disclosure relates to food and beverage products comprising a rare sugar or a combination of rare sugars.

### BACKGROUND

A sugar substitute is a food additive that provides a sweet taste like that of sugar while containing less food energy than sugar (sucrose)-based sweeteners. Artificial and natural high intensity and/or non-nutritive sweeteners may be derived through manufacturing of plant extracts, processed by chemical synthesis, or derived from sugar alcohols. A rare sugar is a monosaccharide that occurs in limited quantities in nature. Rare sugars generally provide little to no nutritional value and can be used as low-calorie sucrose (sugar) alternatives.

### SUMMARY

The present specification is based, at least in part, on the discovery that a combination of two rare sugars, for example allulose and tagatose, can synergistically sweeten food products and beverage products (i.e., providing increased sweetness when used in combination as compared to using exclusively one rare sugar). In particular, applicants have found that infusing food bodies, e.g., cranberries, with a combination of allulose and tagatose increases sweetness while maintaining or improving sensory characteristics including taste, flavor, and texture. Sensory characteristics can also include mouthfeel, plumpness, taste, and orthonasal and retronasal aromas (i.e., aroma & flavor). Further, applicants have found that combining a beverage product with allulose and tagatose increases sweetness while maintaining or improving sensory characteristics. Accordingly, the present specification provides, e.g., a process for infusing food bodies, such as fruit and vegetables, with infusion compositions, e.g., comprising at least two rare sugars, and also provides food products infused with such infusion compositions. Also provided are infusion formulations for performing such methods. The present specification also provides a process for combining the rare sugars with consumable liquids to produce consumable beverage products. Also provided are sugar alternative compositions, e.g., sweetener compositions.

In certain aspects, a (food) product has a food body. The food body includes a food component and an infusion component. The infusion component includes at least two rare sugars including tagatose and allulose.

In some instances, the food body is a fruit body. The fruit body can be a fruit selected from the group consisting of cranberries, blueberries, cherries, grapes, mangos, pineapples, raspberries, blackberries, dates, apples, apricots, lingonberries, tomatoes, huckleberries, chokeberries, figs, gooseberries, elderberries, strawberries, plums, pears, and peaches.

Some infusion components include tagatose and allulose in a ratio of about 1:1, a ratio of about 2:1, or a ratio of about 1:2.

In some instances, the product has a Brix level of about 70 to about 80° Brix, about 72-78° Brix, or about 76 to about 77° Brix. Some products have a Brix level of about 76.3° Brix.

In some embodiments, the product has an acidity of about 1.72%, a moisture level of about 18, and a water activity of about 0.585.

In some cases, the product has a peak positive force of about 6000 grams per second (g/sec) to about 8000 (g/sec), about 7000 (g/sec) to about 7500 (g/sec), about 7200 (g/sec) to about 7350 (g/sec), or about 7250 (g/sec) to about 7300 (g/sec).

Some food bodies are an infused food body having an infused volume. The infused volume is less than a volume of the food body prior to infusion. The infused volume can be about 25% to about 99% of the volume of the food body prior to infusion or about 70% to about 95% of the volume of the food body prior to infusion.

In some instances, the product has a moisture content of about 15% to about 20%, about 16% to about 19%, or about 17.5% to about 18.5%.

In some cases, the product has a water activity of about 0.4 to about 0.7, about 0.55 to about 0.62, or about 0.58 to about 0.59.

In some embodiments, the product has an acidity of about 1% to about 2%, about 1.5% to about 1.95%, or about 1.71% to about 1.73%.

The infusion component can include sucrose. In some infusion components, the at least two rare sugars combined and sucrose may be in a ratio of about 1:2, a ratio greater than 1, or in a ratio less than 1.

In some instances, the product includes sucrose. In some cases, the ratio by weight of sucrose to the at least two rare sugars combined is about 1:1, greater than 1, or less than 1.

In certain aspects, a method is provided to infuse a food product. The method includes providing a food body, providing an infusion formulation having at least two rare sugars including tagatose and allulose, infusing the food body with the infusion formulation, and collecting the food body, to thereby a produce an infused food body.

The at least two rare sugars can include allulose and tagatose and at least a third rare sugar.

In some instances, the method also includes, prior to infusing the food body with the infusion formulation, extracting juice from the food body.

In some cases, the method also includes, prior to infusing the food body with the infusion formulation, extracting about 40% to about 98% of soluble solids from the food body.

In some embodiments, the infusion formulation has a viscosity of about 100 to about 2000 cps.

Some infusion formulations have a dissolved solids content of about 40° Brix to about 70° Brix.

The step of infusing the food body with the infusion formulation includes advancing the food body, in a countercurrent apparatus, along a path while flowing the infusion formulation countercurrently to the advancing food body. The infusion formulation is provided to the countercurrent apparatus at an infusion formulation-to-food body weight/weight ratio of between about 1:1 to about 6:1.

The food body may be a fruit body. The fruit body can be a fruit selected from the group consisting of cranberries, blueberries, cherries, grapes, mangos, pineapples, raspberries, blackberries, dates, apples, apricots, lingonberries, tomatoes, huckleberries, chokeberries, figs, gooseberries, elderberries, plums, prunes, pears, and peaches.

The food body may be a vegetable body. The vegetable body can be a vegetable selected from the group consisting of mushrooms, celery, peppers, carrots, potatoes, cucumbers, corn, onions, peas, and squash.

The step of infusing the food body with the infusion formulation can include infusing the food body with the infusion formulation using a countercurrent apparatus or a vacuum tumbler infuser.

In some cases, the infusion formulation includes by weight/weight, about 10 to about 20% water; about 5 to about 12% cranberry juice concentrate; about 0.5% to about 25% allulose about 0.5% to about 25% tagatose; and about 7.0 to about 16% glycerin.

In some methods, the infusion formulation includes about 7% to about 10% sucrose. The ratio by weight of sucrose to the at least two rare sugars is about 1:1, greater than 1, or less than 1.

In some embodiments, the infusion formulation has a viscosity of about 100 to about 2000 cps.

In some methods, the infusion formulation includes by weight: 23% rare sugars, 12% water and 9.4% cranberry juice concentrate (50° Brix). The 23% rare sugars may include 11.5% tagatose and 11.5% allulose.

In certain aspects, an infusion formulation includes by weight/weight about 10% to about 20% water; about 4% to about 15% juice concentrate; and about 1% to about 25% rare sugars. The rare sugars include allulose and tagatose.

The rare sugars can include allulose, tagatose, and at least one additional rare sugar.

In some infusion formulations, the ratio by weight of the allulose to the tagatose is about 1:1, about 1:2, about 1:3, about 1:4, about 1:5, about 2:1, about 3:1, about 4:1, or about 5:1.

In some infusion formulations, about the ratio by weight of the allulose to the tagatose is about 2:1, about 3:1, about 4:1, or about 5:1.

The infusion formulation can include about 7% to about 10% sucrose. The ratio by weight of sucrose to the allulose and tagatose combined can be about 1:1, greater than 1, or less than 1.

In some embodiments, infusion formulation has a viscosity of about 100 to about 2000 cps, about 100 to about 1000 cps, or about 200 to about 700 cps.

In some cases, the infusion formulation includes about 7.0 to about 16% glycerine.

The juice concentrate may be a cranberry juice concentrate. The juice concentrate can have a dissolved solids content of about 30° Brix to about 70° Brix.

In certain aspects, a beverage includes a consumable liquid, and at least two rare sugars including tagatose and allulose.

In some embodiments, the beverage includes about 0.1% to about 5% of the at least two rare sugars.

In some instincts, the beverage includes about 0.1% to about 3.4% allulose.

In some cases, the beverage includes about 0.1% to about 3.4% tagatose.

In some beverages, about the ratio by weight of the allulose to the tagatose is about 1:1, about 1:2, about 1:3, about 1:4, about 1:5, about 2:1, about 3:1, about 4:1, or about 5:1.

In some beverages, about the ratio by weight of the allulose to the tagatose is about 2:1, about 3:1, about 4:1, or about 5:1.

The beverage may have a dissolvable solids content of about 12.7° Brix, an acidity of about 0.68%, and a pH of about 2.59.

Some beverages have a dissolvable solids content of about 4° Brix to about 15° Brix or about 11° Brix to about 13° Brix.

In some embodiments, the beverage has an acidity of about 0.1% to about 0.8% or about 0.25% to about 0.65%

In some cases, the beverage has a pH of about 2.1 to about 3.1 or about 2.5 to about 2.7.

In some instances, the consumable liquid includes about 4% to about 10% sucrose. The ratio by weight of sucrose to the at least two rare sugars combined can be about 1:1, greater than 1, or less than 1.

Some beverages include about 80% to about 90% water.

In some cases, the consumable liquid is a cranberry juice.

The consumable liquid can be a fruit or vegetable juice, a fruit or vegetable juice concentrate, and/or a fruit or vegetable juice extract.

Some consumable liquids are selected from the group consisting of alcohol, seltzer, cocktail mix, water, sports beverage, soda, carbonated beverage, dairy-based liquid, fruit juice, vegetable juice, or any combination thereof.

In certain aspects, a composition includes at least two rare sugars including allulose and tagatose, and a bulking agent.

In some compositions, the ratio by weight of the allulose to the tagatose is about 1:1, about 1:2, about 1:3, about 1:4, about 1:5, about 2:1, about 3:1, about 4:1, or about 5:1.

In some cases, about the ratio by weight of the allulose to the tagatose in the sweetening composition is about 2:1, about 3:1, about 4:1, or about 5:1.

The composition can have a weight between about 0.25 g and about 5 g.

Some bulking agents include at least one of soluble fibers and sucrose. The ratio by weight of sucrose to the at least two rare sugars is about 1:1, greater than 1, or less than 1.

In certain aspects, a method is provided to produce a beverages. The method includes providing a sweetening composition includes at least two rare sugars including allulose and tagatose, providing a consumable liquid, and mixing the sweetening composition into the consumable liquid to thereby produce a beverage product.

In some methods, mixing the sweetener composition into the consumable liquid includes heating the consumable liquid.

In some methods, the ratio by weight of the allulose to the tagatose in the sweetening composition is about 1:1, about 1:2, about 1:3, about 1:4, about 1:5, about 2:1, about 3:1, about 4:1, or about 5:1.

In some methods, the ratio by weight of the allulose to the tagatose in the sweetening composition is about 2:1, about 3:1, about 4:1, or about 5:1.

In some embodiments, the sweetening composition includes sucrose.

In some instances, the consumable liquid includes sucrose.

In some cases, the consumable liquid is selected from the group consisting of alcohol, seltzer, cocktail mix, water, sports beverage, soda, carbonated beverage, dairy-based liquid, fruit juice, vegetable juice, beverage additive, concentrate, fruit or vegetable concentrate, fruit or vegetable extract, flavoring syrup, or any combination thereof.

In some embodiments, the consumable liquid is about 80% to about 90% water.

In some instances, the beverage has a dissolvable solids content of about 12.7° Brix, an acidity of about 0.68%, and a pH of about 2.59.

In some instances, the beverage has a dissolvable solids content of about 10° Brix to about 15° Brix.

In some instances, the beverage has a dissolvable solids content of about 11° Brix to about 13° Brix.

In some instances, the beverage has an acidity of about 0.5% to about 0.8%.

In some instances, the beverage has an acidity of about 0.64% to about 0.72%.

In some cases, the beverage has a pH of about 2.1 to about 3.1 or about 2.5 to about 2.7.

The step of mixing the sweetening composition into the consumable liquid to thereby produce a beverage product can include heating the consumable liquid.

In certain aspects a food product is provided. The food product includes a food body having a food component and an infusion component. The infusion component has an allulose content of about 5% to about 50%, by weight.

In some embodiments, the food body is a fruit body. The fruit body can be a fruit selected from the group consisting of cranberries, blueberries, cherries, grapes, mangos, pineapples, raspberries, blackberries, dates, apples, apricots, lingonberries, tomatoes, huckleberries, chokeberries, figs, gooseberries, elderberries, strawberries, plums, pears, and peaches. In some cases, the fruit body is a cranberry. The cranberry can have an acidity of about 1.80%, a water activity of about 0.53, and/or a Brix level of at least 77° Brix.

In certain aspects, an infusion formulation is disclosed. The infusion formulation includes, by weight/weight about 5% to about 15% water, about 5% to about 15% cranberry juice concentrate, about 40% to about 60% soluble corn fiber, and about 20% to about 40% allulose.

Some infusion formulations also include 5% to about 15% glycerin, by weight.

In some cases, the infusion formulation has a Brix level of about 60° Brix to about 70° Brix.

In some embodiments, the infusion formulation includes about 0.20% to about 0.40% natural sweetness modifier, by weight.

Accordingly, in one aspect, the invention provides a product that includes a food body comprising a food component and an infusion component. The infusion component can include at least two rare sugars, for example allulose and tagatose. In addition, the infusion component can include additional materials, such as a sugar, fiber, or protein, e.g., a polypeptide, peptide, soluble protein, protein hydrolysate, and/or branched-chain protein. Exemplary proteins include, e.g., gelatin, whey protein, and soy protein. For example, the protein can comprise greater than or equal to 15 amino acid residues, (e.g., greater than or equal to 20, 25, 30, 35, 40, 45, 50, 55, 60, 65, 70, 75, 80, 85, 90, 95, or greater than or equal to 100 amino acid residues). In some instances, the rare sugars can be present in the food body at about 20% to about 50%, or about 30% to about 60%. In some instances, the rare sugars can be present in the food body at greater than 1% by weight. In other instances, the rare sugars can be present in the food body at about 18% to about 25% by weight. In some instances, the total infusion formulation present in the food body is about 20% to about 90% by weight of the total finished food body, e.g., at least about 30% to about 90%, at least about 40% to about 90%, at least about 50% to about 90%, at least about 60% to about 90%, at least about 70% to about 90%, or at least about 80% to about 90%. In some instances, the total infusion formulation present in the food body can be about 90% to about 99% by weight, or greater than 99% by weight.

In some instances, the food body can be a fruit body. The fruit body can be, e.g., a fruit selected from the group consisting of cranberry, blueberry, cherry, grape, mango, pineapple, raspberry, blackberry, date, apple, apricot, lingonberry, tomato, huckleberry, chokeberry, fig, gooseberry, elderberry, plum, prune, pear, and peach, among others. In some embodiments, an infusion formulation can include, in addition to at least two rare sugars, e.g., water, one or more types of sugar (e.g., sucrose, glucose, and/or fructose), a juice (e.g., cranberry juice and/or blueberry juice), a fruit syrup (e.g., pineapple syrup and/or agave nectar), honey, an organic acid (e.g., citric, tartaric, quinic, fumaric, malic and/or lactic acid) and/or a humectant (e.g., glycerol). In some embodiments, an infusion formulation can include, e.g., ribose, sucrose, dextrose, maltose, maltotriose, cellobiose, or sugar alcohols such as sorbitol, lactitol, xylitol etc., whey or soy protein hydrolysates, non-nutritive sweeteners, and/or emulsifiers such as mono and di-glycerides, polysorbates, etc. For example, an infusion formulation can include by weight/weight about 10% to about 20% water, about 38% to about 50% soluble corn fiber, about 5% to about 35% sugar such as sucrose (e.g., about 7% to about 10%), about 4% to about 15% (e.g., about 5% to about 12%) cranberry juice concentrate (50° Brix), about 0% to about 2% non-nutritive/low-calorie sweetener, and optionally, about 7.0% to about 16% glycerine. In some embodiments, the infusion formulation can have a viscosity of about 100 to about 2000 cps, e.g., about 100 to about 1000 cps, e.g., about 300 to about 1000 cps, e.g., about 400 to about 2000 cps, e.g., about 1000 to about 2000 cps, and have a content of about 10 to 65° Brix. In some embodiments, the infused food bodies, e.g., infused cranberries, can have an infused solids content of at least or about 40° Brix, e.g., at least or about 60, 65, 70, or at least or about 75° Brix, before drying of the food body

As used herein, the term “food body” means a unit of food, e.g., a fruit, that retains its native structure at least in part, e.g., a fruit hull that is uncomminuted. A food body retains sufficient native structure to allow the food body to be infused with, and retain within its structure, a substantial amount of an infusion formulation described herein after the food body is removed from the infusion formulation (i.e., the body is capable of retaining infusion formulation at least about 0.1%, e.g., at least about 0.5%, at least about 1%, at least about 2%, at least about 5%, at least about 8%, at least about 10%, at least about 20% or at least about 30% (e.g., up to about 90%) total weight of the body after infusion). Thus, the term “food body” does not include purees, jellies,” or juices. An exemplary body is a cranberry hull that has been subjected to extraction in a counter current extraction (CCE) apparatus, e.g., as described in U.S. Pat. No. 5,320,861. The terms “fruit body,” and vegetable body,” are types and examples of food bodies.

As used herein, the term “sweetener composition” means a unit of edible or drinkable additive to a liquid or food, e.g., powdered sugar alternatives, granular sugar alternatives, liquid sweeteners, that includes at least two rare sugars, for example allulose and tagatose. The sweetener composition generally includes at least two rare sugars and, in some cases, bulking agent, e.g., soluble corn fiber, soluble tapioca fiber, malto-oligosaccharides, fructo-oligosaccharides, sugar, high intensity sweeteners (e.g., sucralose, stevia, or other natural flavors).

As used herein, the term “food product” means a unit of edible food, e.g., health bars, granola bars, nut butters, candy, baked goods, dairy based products, fruits, chewable snack, vegetables, powdered extracts, sugar alternatives, seasoning, cheeses, mints, gum, crackers, diary alternative products, jellies, powders, that includes at least two rare sugars, for example allulose and tagatose. Thus, the term “food product” does not include beverages, water products, purees, alcohol products, or broths or any food that does not include at least two rare sugars, e.g., allulose and tagatose. A “food product” may include a “food body” and/or a “sweetener composition” as previously described.

As used herein, the term “consumable liquid” means a unit of ingestible or drinkable liquid, e.g., alcoholic beverages, water-based liquids, flavored waters, carbonated liquids, sodas, broths, juices, purees, liquid extracts, syrups, milk, milk alternatives, smoothies, not comprising a combination of at least two rare sugars prior to addition of the rare sugars in accordance with the methods described herein. Thus the term “consumable liquid” does not include foods, or any beverage already including at least two rare sugars, e.g., allulose and tagatose.

As used herein, the term “consumable beverage product” means a unit of a drinkable liquid comprising at least two rare sugars, for example allulose and tagatose. The consumable beverage product can be any sweetened liquid, e.g., alcoholic beverages, water-based liquids, flavored waters, carbonated liquids, sodas, broths, juices, purees, liquid extracts, syrups, milk, milk alternatives, smoothies, liquid sweeteners. Thus, the term “beverage product” does not include edible foods or any consumable liquid that does not include at least two rare sugars, e.g., allulose and tagatose. A “consumable beverage product” may include a “consumable liquid” or a “sweetener composition” as previously described.

Unless otherwise defined, all technical terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

Other features and advantages of the invention will be apparent from the following detailed description and figure, and from the claims.

### DESCRIPTION OF DRAWINGS

FIG. 1 depicts a flow diagram illustrating an exemplary procedure for infusing a cranberry with at least two rare sugars.

FIG. 2 depicts a flow diagram illustrating an exemplary procedure for producing a beverage product with at least two rare sugars.

Like reference symbols in the various drawings indicate like elements.

### DETAILED DESCRIPTION

This disclosure relates to a sweetener composition that includes a combination of rare sugars, for example allulose and tagatose. The disclosure further relates to methods of making a food product and consumable beverage product. The sweetener composition can include a first rare sugar (allulose) and a second rare sugar (tagatose), which together provide a synergistic sweetening effect. The combination of allulose and tagatose has an increased sweetness as compared to the same amount of allulose alone or the same amount of tagatose alone in food products, infused food products, sweetener compositions, and beverage products. The use of the combination of rare sugars in food products can provide a balanced sweet/tart profile and a stronger upfront sweetness and flavor intensity, while maintaining the substantially the same flavor profile and texture as compared to food products comprising only standard sugars, e.g., sucrose.

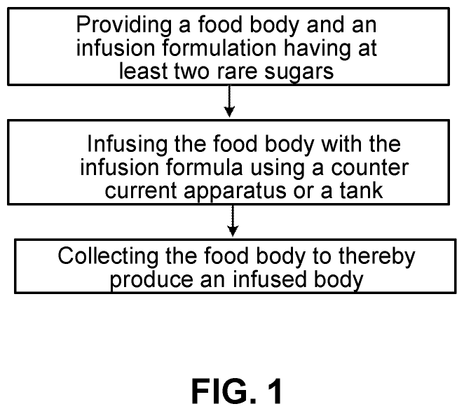

A flow diagram is shown in FIG. 1 of an exemplary infusion procedure, wherein infusion formulations comprising rare sugars described herein can be infused into food bodies, such as cranberries. In this exemplary procedure, a commercially available countercurrent apparatus is used, such as a countercurrent apparatus described in U.S. Pat. No. 5,320,861. However, it will be understood by skilled practitioners that other infusion processes and equipment (including but not limited to tank systems, vacuum tumbler systems and co-current systems) can be used. The process will be described for use with cranberries, although it may be adapted for use with many other types of fruit such as blueberries, cherries or grapes.

Infusion Formulations

Included in the present invention are various infusion formulations. An infusion formulation may contain at least two rare sugars. The term “rare sugar” is a term used in the art and recognized by skilled practitioners as a sucrose alternative, for example allulose and tagatose. Generally, rare sugars are low calorie chemicals that have little to no nutritional value. The rare sugars can be used in place of or in conjunction with sucrose to sweeten any ingestible product (e.g., any food or beverage).

In some instances, the infusion formulation can have, by weight, about 0.1% to about 50% rare sugars, For example, the infusion formulation can have, by weight, about 0.1% to about 5% rare sugars, e.g., about 1% to about 10% rare sugars, e.g., about 1% to about 20% rare sugars, e.g., about 1% to about 30% rare sugars, e.g., about 1% to about 40% rare sugars, e.g., about 10% to about 20% rare sugars, e.g., about 10% to about 30% rare sugars, e.g., about 10% to about 45% rare sugars, e.g., about 20% to about 25% rare sugars, e.g., about 20% to about 30% rare sugars, e.g., about 30% to about 40% rare sugars, e.g., about 40% to about 50% rare sugars, e.g., about 0.5% to about 1% rare sugars, e.g., about 0.1% to about 2% rare sugars, e.g., about 0.1% to about 3% rare sugars, e.g., about 2% to about 3% rare sugars, e.g., about 3% to about 5% rare sugars, e.g., about 4% to about 6% rare sugars, e.g., about 8% to about 15% rare sugars, e.g., about 15% to about 20% rare sugars, e.g., about 20 to about 40% rare sugars, e.g., or about 1% to about 8%.

An infusion formulation of the present invention can have, by weight, 0.1% to about 50% allulose. For example, an infusion formulation can have, by weight, about 0.1% to about 5% allulose, e.g., about 0.1% to about 3.4% allulose, e.g., about 1% to about 10% allulose, e.g., about 1% to about 20% allulose, e.g., about 1% to about 30% allulose, e.g., about 1% to about 40% allulose, e.g., about 10% to about 20% allulose, e.g., about 10% to about 30% allulose, e.g., about 10% to about 45% allulose, e.g., about 20% to about 25% allulose, e.g., about 20% to about 30% allulose, e.g., about 30% to about 40% allulose, e.g., about 40% to about 50% allulose, e.g., about 0.5% to about 1% allulose, e.g., about 0.1% to about 2% allulose, e.g., about 0.1% to about 3% allulose, e.g., about 2% to about 3% allulose, e.g., about 3% to about 5% allulose, e.g., about 4% to about 6% allulose, e.g., about 2% to about 6% allulose, e.g., about 8% to about 15% allulose, e.g., about 15% to about 20% allulose, e.g., about 20 to about 40% allulose, or about 1% to about 8% allulose.

An infusion formulation of the present invention can have, by weight, 0.1% to about 50% tagatose. For example, an infusion formulation can have, by weight, about 0.1% to about 5% tagatose, e.g., about 0.1% to about 3.4% tagatose, e.g., about 1% to about 10% tagatose, e.g., about 1% to about 20% tagatose, e.g., about 1% to about 30% tagatose, e.g., about 1% to about 40% tagatose, e.g., about 10% to about 20% tagatose, e.g., about 10% to about 30% tagatose, e.g., about 10% to about 45% tagatose, e.g., about 20% to about 25% tagatose, e.g., about 20% to about 30% tagatose, e.g., about 30% to about 40% tagatose, e.g., about 40% to about 50% tagatose, e.g., about 0.5% to about 1% tagatose, e.g., about 0.1% to about 2% tagatose, e.g., about 0.1% to about 3% tagatose, e.g., about 2% to about 3% tagatose, e.g., about 3% to about 5% tagatose, e.g., about 4% to about 6% tagatose, e.g., about 2% to about 6% tagatose, e.g., about 8% to about 15% tagatose, e.g., about 15% to about 20% tagatose, e.g., about 20 to about 40% tagatose, e.g., or about 1% to about 8%.

The infusion formulations of the present invention can have e.g., a ratio of allulose to tagatose, by weight, of about 1:25 to about 25:1. For example, the infusion formulation can have a ratio of allulose to tagatose, by weight, of about 1:1, e.g., a ratio of allulose to tagatose of about 1:2, e.g., a ratio of allulose to tagatose of about 1:3, e.g., a ratio of allulose to tagatose of about 1:4, e.g., a ratio of allulose to tagatose of about 1:5, e.g., a ratio of allulose to tagatose of about 1:6, e.g., a ratio of allulose to tagatose of about 1:7, e.g., a ratio of allulose to tagatose of about 1:8, e.g., a ratio of allulose to tagatose of about 1:9, e.g., a ratio of allulose to tagatose of about 1:10, e.g., a ratio of allulose to tagatose of about 1:15, e.g., a ratio of allulose to tagatose of about 1:20, e.g., a ratio of allulose to tagatose of about 1:25, e.g., a ratio of allulose to tagatose of about 2:1, e.g., a ratio of allulose to tagatose of about 3:1, e.g., a ratio of allulose to tagatose of about 4:1, e.g., a ratio of allulose to tagatose of about 5:1, e.g., a ratio of allulose to tagatose of about 6:1, e.g., a ratio of allulose to tagatose of about 7:1, e.g., a ratio of allulose to tagatose of about 8:1, e.g., a ratio of allulose to tagatose of about 9:1, e.g., a ratio of allulose to tagatose of about 10:1 e.g., a ratio of allulose to tagatose of about 1:15, e.g., a ratio of allulose to tagatose of about 1:20, or e.g., a ratio of allulose to tagatose of about 1:25.

In some instances, the formulation can have a viscosity of between about 100 to about 2000 cps. For example, the formulation can have a viscosity of about 100 to about 1000, e.g., about 500 to about 1500 cps, e.g., about 1000 to about 2000 cps, e.g., about 1000 to about 1500 cps. In some embodiments, the infusion formulation can have a viscosity of about 1000 cps, about 1500 cps, or about 2000 cps. In some embodiments, the formulation can have a viscosity as low as 25 cps, e.g., when infusing at higher temperature, e.g., between 50° F. to 140° F. (e.g., about 63° F.).

The infusion formulations of the present invention can have a dissolved solids content of, e.g., about 10 to about 65° Brix, e.g., about 40 to about 80° Brix, e.g., about 40 to about 75° Brix, or about 50° Brix to about 70° Brix, e.g., about 55° Brix to about 65° Brix. For example, in some embodiments, the infusion formulation has a dissolved solids content of about 45° Brix to 55° Brix, about 60° Brix, about 65° Brix, or about 65° Brix to about 75° Brix.

Formulations can include an aqueous solution, such as water. Further, infusion formulations can include one or more standard sugars. Sugars that can be included in a formulation include, for example, glucose, ribose, sucrose, fructose, dextrose, maltose, maltotriose, and/or cellobiose. Alternatively or in addition, a sugar alcohol can be included. Exemplary sugar alcohols include sorbitol, lactitol, xylitol, mannitol, maltitol, isomalt, glycerol, erythritol, and/or arabitol. Alternatively or in addition, a juice can be included. Skilled practitioners will appreciate that any juice can be included depending upon the characteristics desired for the product. For example, cranberry juice, blueberry juice, and/or cherry juice can be added, e.g., in concentrated form. Alternatively or in addition, the formulation can include a non-nutritive sweetener such as Stevia, Aspartame, Sucralose, Luo Han Guo, and/or Erythritol.

Alternatively or in addition, the formulation can include soluble polysaccharides such as starch, hemi-celluloses or other hydrocolloids, such as pectin or other gums (e.g., Guar, Gum Acacia, Locust Bean, Carob, Xanthan, Gellan, Konjac, Carrageenan, Gum Karaya), or a soluble protein or protein hydrolysate. In some embodiments, the formulations can include mixtures of infusible rare sugars described herein, e.g., any of the rare sugars described herein and/or fused/hybrid forms of any of the rare sugars described herein.

Skilled practitioners will appreciate that the infusion formulation can include any number of other useful ingredients, e.g., materials to improve the appearance, taste, or nutritional properties of the product. For example, the infusion formulation may include flavoring other than sugar, e.g., spices such as cinnamon, mint, and/or peppermint, may include nutritionally valuable components, such as vitamins, e.g. ascorbic acid, and/or minerals, e.g. iron and/or calcium, or flavor modifiers, e.g., such as a natural sweetness modifier.

Skilled practitioners will also appreciate that in order to improve the mouth feel and/or the texture of the food body, or to improve the functionality of the food body, plasticizers/humectants such as glycerol, erythritol, sorbitol, fructose, dextrose, fructo-oligosaccharides (FOS), galacto-oligosaccharides (GOS), monoglycerides, propylene glycol, lactic acid, and/or glyceryl triacetate can be added to the infusion formulation.

In some instances, a first infusion formulation and a second infusion formulation are used to infuse the food body with at least two rare sugars. The first and second infusion formulations are substantially similar to the combined rare sugar infusion formulation, described previously, however, here the first infusion formulation includes a first rare sugar and the second formulation includes a second rare sugar. For example, the first infusion formulation can include allulose and the second infusion formulation can include tagatose. In some cases the first and/or the second infusion formulations include more than one rare sugar. Infusing the food body with a first and second infusion formulation may reduce osmotic shock.

In some instances, the first infusion formulation can have, by weight, about 0.1% to about 50% rare sugars, For example, the first infusion formulation can have, by weight, about 0.1% to about 5% rare sugars, e.g., about 1% to about 10% rare sugars, e.g., about 1% to about 20% rare sugars, e.g., about 1% to about 30% rare sugars, e.g., about 1% to about 40% rare sugars, e.g., about 10% to about 20% rare sugars, e.g., about 10% to about 30% rare sugars, e.g., about 10% to about 45% rare sugars, e.g., about 20% to about 25% rare sugars, e.g., about 20% to about 30% rare sugars, e.g., about 30% to about 40% rare sugars, e.g., about 40% to about 50% rare sugars, e.g., about 0.5% to about 1% rare sugars, e.g., about 0.1% to about 2% rare sugars, e.g., about 0.1% to about 3% rare sugars, e.g., about 2% to about 3% rare sugars, e.g., about 3% to about 5% rare sugars, e.g., about 4% to about 6% rare sugars, e.g., about 8% to about 15% rare sugars, e.g., about 15% to about 20% rare sugars, e.g., about 20 to about 40% rare sugars, e.g., or about 1% to about 8%.

A first infusion formulation of the present invention can have, by weight, 0.1% to about 50% allulose. For example, a first infusion formulation can have, by weight, about 0.1% to about 5% allulose, e.g., about 0.1% to about 3.4% allulose, e.g., about 0.5% to about 25% allulose, about 1% to about 10% allulose, e.g., about 1% to about 20% allulose, e.g., about 1% to about 30% allulose, e.g., about 1% to about 40% allulose, e.g., about 10% to about 20% allulose, e.g., about 10% to about 30% allulose, e.g., about 10% to about 45% allulose, e.g., about 20% to about 25% allulose, e.g., about 20% to about 30% allulose, e.g., about 30% to about 40% allulose, e.g., about 40% to about 50% allulose, e.g., about 0.5% to about 1% allulose, e.g., about 0.1% to about 2% allulose, e.g., about 0.1% to about 3% allulose, e.g., about 2% to about 3% allulose, e.g., about 3% to about 5% allulose, e.g., about 4% to about 6% allulose, e.g., about 2% to about 6% allulose, e.g., about 8% to about 15% allulose, e.g., about 15% to about 20% allulose, e.g., about 20 to about 40% allulose, or about 1% to about 8% allulose.

Further, in some instances, the second infusion formulation can have, by weight, about 0.1% to about 50% rare sugars, For example, the second infusion formulation can have, by weight, about 0.1% to about 5% rare sugars, e.g., about 1% to about 10% rare sugars, e.g., about 1% to about 20% rare sugars, e.g., about 1% to about 30% rare sugars, e.g., about 1% to about 40% rare sugars, e.g., about 10% to about 20% rare sugars, e.g., about 10% to about 30% rare sugars, e.g., about 10% to about 45% rare sugars, e.g., about 20% to about 25% rare sugars, e.g., about 20% to about 30% rare sugars, e.g., about 30% to about 40% rare sugars, e.g., about 40% to about 50% rare sugars, e.g., about 0.5% to about 1% rare sugars, e.g., about 0.1% to about 2% rare sugars, e.g., about 0.1% to about 3% rare sugars, e.g., about 2% to about 3% rare sugars, e.g., about 3% to about 5% rare sugars, e.g., about 4% to about 6% rare sugars, e.g., about 8% to about 15% rare sugars, e.g., about 15% to about 20% rare sugars, e.g., about 20 to about 40% rare sugars, e.g., or about 1% to about 8%.

A second infusion formulation of the present invention can have, by weight 0.1% to about 50% tagatose. For example, a second infusion formulation can have, by weight, about 0.1% to about 5% tagatose, e.g., about 0.1% to about 3.4% tagatose, about 0.5% to about 25% tagatose, e.g., about 1% to about 10% tagatose, e.g., about 1% to about 20% tagatose, e.g., about 1% to about 30% tagatose, e.g., about 1% to about 40% tagatose, e.g., about 10% to about 20% tagatose, e.g., about 10% to about 30% tagatose, e.g., about 10% to about 45% tagatose, e.g., about 20% to about 25% tagatose, e.g., about 20% to about 30% tagatose, e.g., about 30% to about 40% tagatose, e.g., about 40% to about 50% tagatose, e.g., about 0.5% to about 1% tagatose, e.g., about 0.1% to about 2% tagatose, e.g., about 0.1% to about 3% tagatose, e.g., about 2% to about 3% tagatose, e.g., about 3% to about 5% tagatose, e.g., about 4% to about 6% tagatose, e.g., about 2% to about 6% tagatose, e.g., about 8% to about 15% tagatose, e.g., about 15% to about 20% tagatose, e.g., about 20 to about 40% tagatose, e.g., or about 1% to about 8%.

First and second infusion formulations of the present invention can have, e.g., a ratio of allulose in the first infusion formulation to tagatose in the second infusion formulation, by weight, of about 1:25 to about 25:1. For example, the first and second infusion formulations can have a ratio of allulose in the first infusion formulation to tagatose in the second infusion formulation, by weight, of about 1:1, e.g., a ratio of allulose to tagatose of about 1:2, e.g., a ratio of allulose to tagatose of about 1:3, e.g., a ratio of allulose to tagatose of about 1:4, e.g., a ratio of allulose to tagatose of about 1:5, e.g., a ratio of allulose to tagatose of about 1:6, e.g., a ratio of allulose to tagatose of about 1:7, e.g., a ratio of allulose to tagatose of about 1:8, e.g., a ratio of allulose to tagatose of about 1:9, e.g., a ratio of allulose to tagatose of about 1:10, e.g., a ratio of allulose to tagatose of about 1:15, e.g., a ratio of allulose to tagatose of about 1:20, e.g., a ratio of allulose to tagatose of about 1:25, e.g., a ratio of allulose to tagatose of about 2:1, e.g., a ratio of allulose to tagatose of about 3:1, e.g., a ratio of allulose to tagatose of about 4:1, e.g., a ratio of allulose to tagatose of about 5:1, e.g., a ratio of allulose to tagatose of about 6:1, e.g., a ratio of allulose to tagatose of about 7:1, e.g., a ratio of allulose to tagatose of about 8:1, e.g., a ratio of allulose to tagatose of about 9:1, e.g., a ratio of allulose to tagatose of about 10:1 e.g., a ratio of allulose to tagatose of about 1:15, e.g., a ratio of allulose to tagatose of about 1:20, or e.g., a ratio of allulose to tagatose of about 1:25.

### Infusion Formulation Examples

General exemplary infusion formulations are described below, which may be used in any of the methods described herein and do not limit the scope of the invention described in the claims. All percentage values are provided on a weight/weight basis:

Formulation A Infusion Syrup

| | | |
|---|---|---|
| 1. | Allulose | 11.1% w/w |
| 2. | Tagatose | 11.1% w/w |
| 3. | Soluble Com Fiber | 56.4% w/w |
| 4. | Cranberry Concentrate, 50° Brix | 9.4% w/w |
| 5. | Water | 12.0% w/w |

### Exemplary Infusion Method

In an exemplary infusion method, cranberries that have been subjected to an extraction method to extract juice and which retain about 4% to about 30% of their soluble solids are supplied to a countercurrent infusion apparatus (CCI). A CCI useful in the present methods includes an elongate trough-shaped housing with a helical screw conveyor intermittently rotated by a motor means, connected to a shaft on its longitudinal axis. The housing has an inlet for the introduction of material to be infused, such as cranberries, and an outlet at the other end of the trough housing is provided for removal of infused fruit. The inlet is disposed above the lower end of the screw, which is inclined slightly upwardly at an angle, e.g., of 2 to 6 degrees. The trough temperature may be controlled (e.g., by heating or cooling with a circulating water jacket positioned about the trough) to control the process temperature. Alternatively or in addition, the temperature of the fruit or the infusion formulation may be preselected prior to introduction to the extractor. The screw conveyor is operated by intermittently reversing the direction of rotation of the screw. The reversal helps the relatively compacted mass of matter being infused to be opened up enhancing the penetration of infusion formulation. Other details of a suitable CCI and methods are described in U.S. Pat. No. 4,363,264, the entire contents of which are hereby incorporated by reference. Commercially available units (e.g., CCE Model 1200, Millerbernd Systems, Winsted, Minn.) may be modified and operated with beneficial results. Infused fruit exits CCI through outlet and can optionally be moved to dryer, which passes forced air through the infused fruit product to remove water. Drying temperature is typically in the range of about 180° F. to 200° F. for about 120 minutes using a conventional forced air fruit dryer. The dried, infused fruit may optionally be passed to an oiler wherein vegetable oil or the like is applied to the fruit product to enhance flowability. The final dried infused product is collected and may be bulk packaged or packaged in pouches, e.g., for retail sale.

Liquid input to the CCI apparatus is an infusion formulation, e.g., an infusion formulation described herein comprising at least one or more rare sugars. The infusion formulation can be provided to the infuser by any method known in the art. For example, the formulation can be provided initially from a batching tank which provides the formulation to a holding tank. Formulation flows from holding tank to the CCI via a charging line, which charges infusion formulation into the housing. Spent infusion formulation flows out of the CCI via a discharge line. The infusion formulation can be provided from a continuous process loop that mixes spent infusion formulation that has run through the CCI apparatus with fresh infusion formulation from a supply to produce a refreshed infusion formulation. For example, the infusion formulation can run through discharge line to evaporator feed tank, which feeds spent infusion formulation to evaporator. After evaporation, infusion formulation flows to CCI feed tank. The infusion formulation from CCI feed tank then mixes with fresh infusion formulation from hold tank, and is then fed back into CCI to complete the process loop.

Infusion is carried out in the CCI apparatus under conditions that allow efficient infusion of infusion syrup, e.g., rare sugars, for example allulose and tagatose, either individually or in combination, into the fruit. Skilled practitioners will appreciate that the combination of allulose and tagatose infusion formulations can allow a higher sweetness profile than allulose or tagatose alone, while maintaining mouthfeel and plumpness. Skilled practitioners will also appreciate that rare sugars need not be infused into a food body, e.g., a cranberry, simultaneously or together in the same infusion formulation. Rather, for example, two infusion formulations, each including only one of the rare sugars, can be infused into the food body sequentially. For example, a fruit or vegetable body can be infused with a first infusion formulation, including allulose, then later infused with a second infusion formulation, including tagatose by the same or a different CCI.

The residence time of the fruit in the CCI apparatus can vary as necessary. Skilled practitioners will appreciate that increased residence times may allow for increased levels of infusion, e.g., depending upon the equipment used in the methods. In some embodiments, the residence time is about 50 to about 80 minutes, e.g., about 40 to about 60 minutes, or about 45 to about 55 minutes. In other embodiments, a residence time of greater than 1 hour, e.g., about 1 hour to about 12 hours may be used, e.g., about 2 hours to about 3 hours, e.g., about 2 hours, may be used, as can a residence time of about 8 hours to about 10 hours.

The temperature at which infusion is performed may be close to room temperature, e.g., about 65° F. to about 80° F. Skilled practitioners will appreciate that lower or higher temperatures may be used in certain situations. For example, infusions may be performed at higher temperatures, e.g., about 120° F. to about 130° F. As discussed above, the temperature at which infusion is performed can be controlled by any means known in the art, e.g., by controlling the trough temperature, the fruit temperature, and/or the infusion formulation temperature.

In some instances, the fruit body is infused using a tank rather than a counter current apparatus. In an exemplary method, a food body is added to a tank containing a concentrated infusion formulation (e.g., an infusion formulation bath). The suspended fruit bodies and infusion formulation are stirred. Due to the greater amount of dissolved solids in the bath versus that present in the fruit bodies, osmotic exchange takes place resulting in the infusion of the infusion formulation into the fruit body. During osmosis, the infusion formulation diffuses inwardly into the fruit while water in the fruit body diffuses outwardly into the infusion formulation.

In some cases, a fruit body can be infused with a first infusion formulation, having a first rare sugar (e.g., one of allulose or tagatose) in a countercurrent apparatus. The fruit or vegetable body can then be infused with a second rare sugar (e.g., the other one of allulose or tagatose) using a tank, prior to or after infusion of the first infusion formulation.

Further, in some instances, a fruit body can be infused, in a tank, with a first infusion having a first rare sugar (e.g., one of allulose or tagatose). The fruit or vegetable body can then be infused with a second rare sugar (e.g., the other one of allulose or tagatose) using the same or a different tank, prior to or after infusion of the first infusion formulation.

Food Products

The methods described herein can provide a large number of food products, e.g., infused food products. A food product can include, e.g., a food body, e.g., a fruit body, a vegetable or portion thereof, a dairy based product (e.g., yogurt, ice cream, cheese, butter, sour cream), a dairy alternative product, a sorbet, a cracker, a baked good (e.g., rolls, croissants, cakes, cookies, bagels), seasoning, a chewable snack, mints, gum, jellies, and powders, into which at least two rare sugars, e.g., allulose and tagatose, are infused, or to which at least two rare sugars (e.g., allulose and tagatose) have been added.

An infused product typically includes a food component (i.e., the natural and/or endogenous material of the infused food product, e.g., a fruit hull, such as a cranberry or blueberry hull) and an infusion component (i.e., one or more exogenously-added infusion formulation(s) described herein) that comprises at least two rare sugars. In some instances, the infused product will include a fruit (e.g., cranberry or blueberry) hull that comprises within it one or more infusion formulation(s) (e.g., two infusion formulations), wherein the infusion formulation(s) occupies (e.g., partially or completely) one or more voids left within the fruit hull as a result of the fruit being treated in an extraction process (e.g., squeezing, subcritical water extraction, and/or countercurrent extraction) prior to being treated with the presently-described infusion process.

For example, any type of fruit body that can be infused with an infusion formulation and retain a substantial amount of the infusion formulation within the body (i.e., retain infusion formulation at least about 0.1%, e.g., at least about 1%, at least about 2%, at least about 5%, at least about 8%, at least about 10%, at least about 20% or at least about 30% total weight of the food body after infusion) after removal of the food body from the infusion formulation, can be used and is included in the invention in its infused form.

The infused food bodies can be, e.g., infused dried fruit, for example sweetened dried cranberries. In some embodiments, the infused food bodies, e.g., infused cranberries, will have infused solids content of at least or about 40° Brix, e.g., at least or about 50° Brix, at least or about 55° Brix, at least or about 60° Brix, at least or about 65° Brix, at least or about 70° Brix, or at least or about 75° Brix, before drying of the food body.

In some instances, the infused food products may be characterized by including an at least two rare sugars (e.g., allulose and tagatose) content of at least or about 1% by weight. For example, the infused food bodies can have at least two rare sugars content ranging from about 1% to about 65%, e.g., about 1% to about 5%, about 2% to about 5%, about 3% to about 5%, about 1% to about 3%, about 0.5% to about 2.5%, about 0.5% to about 3.5%, about 2% to about 5%, about 1% to about 10, about 1% to about 8%, 1% to about 7%, about 5% to about 50% or about 10% to about 20%, by weight. In some embodiments, the infused food bodies can have at least two rare sugars content of about 1.5% to about 3.5%, e.g., about 2.0% to about 2.5%, e.g., a content of at least or about 1% by weight. In certain embodiments, the food products include food bodies that are infused with two rare sugars. However, it is also contemplated that mixed infusions can be performed, e.g., wherein more than two rare sugars are infused, e.g., a mixture of any combination of rare sugars described herein is infused, or wherein fused/hybrid forms of infusible rare sugars, e.g., the rare sugars described herein, are infused. In these instances, mixed infusion formulations comprising rare sugars, standard sugar, and mixtures thereof may be used. Infused food bodies, comprising a mixture of rare sugars wherein the total rare sugars content is as described herein, are also within the present invention. In some cases, the infusion formulation includes at least two rare sugars and sucrose. In some instances, the infusion formulation may comprise from at least about 0.1%, e.g., at least about 0.5%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 30%, at least about 40%, at least about 50%, at least about 60%, at least about 70%, at least about 80%, at least about 90%, or at least about 98%, of the total weight of the infused food body. The infused food bodies can be in dried, semi-dried, or non-dried form.

The infusion component and/or the food components can further include sucrose, for example about 0% to about 60% sucrose. The ratio of sucrose to the at least two rare sugars can be about 1 (i.e., about equal sucrose to rare sugars), greater than one, (more sucrose than rare sugars), or less than one (more rare sugars than sucrose). The sucrose may be naturally derived sucrose present the food component, for example naturally occurring sugars in fruits, added to the food components, and/or may be part of the infusion component. As shown in the Examples, the at least two rare sugars, e.g., allulose and tagatose, combined with the food product, produce a synergistic phenomenon in which the sweetness profile of the product is increased as compared to using only allulose or only tagatose. Further, the inclusion of both allulose and tagatose in a food body with sugar, reduces the amount of sugar needed to create a certain sweetness profile. The addition of the combination of allulose and tagatose maintains the mouthfeel and structure (plumpness) of the infused food product, such as cranberries, without any added grittiness or noticeable after taste.

In one example the food product, e.g., an infused cranberry, can have an acidity of about 1.72%, a moisture level of about 18, and a water activity of about 0.585.

The food product, e.g., the infused food body, can in some instances include allulose and tagatose in a ratio by weight of allulose to tagatose, of about 1:25 to about 25:1, e.g., a ratio of about 2:1, in a ratio of about 3:1, in a ratio of about 4:1, in a ratio of about 5:1, in a ratio of about 6:1, in a ratio of about 7:1, in a ratio of about 8:1, in a ratio of about 9:1, in a ratio of about 10:1, in a ratio of about 15:1, in a ratio of about 20:1 in a ratio of about 25:1, in a ratio of about 1:2, in a ratio of about 1:3, in a ratio of about 1:4, in a ratio of about 1:5, in a ratio of about 1:6, in a ratio of about 1:7, in a ratio of about 1:8, in a ratio of about 1:9, in a ratio of about 1:10, in a ratio of about 1:15 in a ratio of about 1:20, or in a ratio of about 1:25.

The food product, e.g., the infused food body, can in some instances have a Brix level of about 70 to about 80° Brix, e.g., about 72 to about 78° Brix, about 73 to about 77° Brix, about 74 to about 76.5, about 73 to about 80° Brix, about 75 to about 78° Brix, ° Brix, about 76 to about 77° Brix, e.g., about 70° Brix, about 71° Brix, about 72° Brix, about 73° Brix, about 74° Brix, about 75° Brix, about 76° Brix, about 77° Brix, about 78° Brix, about 79° Brix, about 80° Brix, about 76.1° Brix, about 76.2° Brix, about 76.3° Brix, about 76.4° Brix, about 76.5° Brix, about 76.6° Brix, or about 76.7° Brix.

The food product, e.g., the infused food body, can in some instances have a peak positive force of about 6000 g/sec to about 8000 g/sec, e.g., about 7000 g/sec to about 7500 g/sec, about 7200 g/sec to about 7350 g/sec, or about 7250 g/sec to about 7300 g/sec. The peak positive force can be measured by a texture analyzer, for example the TA XT Plus Texture Analyzer. The texture analyzer measured applied pressure of spikes into a product or food body to mimic the bite of a consumer's front teeth.

The food product, e.g., the infused food body, can in some instances have a moisture content of about 15% to about 20%, about 16% to about 19%, or about 17.5% to about 18.5%.

The food product, e.g., the infused food body, can in some instances have a water activity of about 0.4 to about 0.7, about 0.55 to about 0.62, or about 0.58 to about 0.59. The water activity is the partial vapor pressure of water in a solution divided by the standard state partial vapor pressure of water. In the field of food science, the standard state is most often defined as the partial vapor pressure of pure water at the same temperature.

The food product, e.g., the infused food body, can in some instances have an acidity of about 1% to about 2%, about 1.5% to about 1.95%, or about 1.71% to about 1.73%.

The food product, e.g., the infused food body, can in some instances have a piece count of about 0.60 pieces about 0.80 pieces/g, about 0.65 pieces/g, or about 0.71 pieces/g. The food product, e.g., the infused food body, has an infused volume.

The food body, prior to infusion, has a volume. Generally, the infused volume (after infusion) is less than the volume of the food body prior to processing (e.g., infusion, drying, and/or extraction). The infused volume, in some instances can be about 25% to about 99% of the volume of the food body prior to infusion, e.g., about 25% to about 80% of the volume of the food body prior to infusion, e.g., about 25% to about 75% of the volume of the food body prior to infusion, e.g., about % to about 90% of the volume of the food body prior to infusion, e.g., about 50% to about 75% of the volume of the food body prior to infusion, e.g., about 70% to about 90% of the volume of the food body prior to infusion, e.g., about 75% to about 85% of the volume of the food body prior to infusion, or e.g., about 40% to about 80% of the volume of the food body prior to infusion. In some instances, the infused volume of the infused food is equal to or greater than the volume than the volume of the food body prior to infusion. In particular, any type of food body, e.g., cranberries, infused according to a process described herein, such as cranberries infused with at least two rare sugars, e.g., allulose and tagatose, are within the present invention. Exemplary fruit bodies include, but are not in any way limited to, cranberries, blueberries, cherries, grapes, mangos, pineapples, raspberries, blackberries, dates, apples, apricots, lingonberries, tomatoes, huckleberries, chokeberries, figs, gooseberries, elderberries, plums, prunes, pears, peaches, bananas, mangos, black berries, and the like. Skilled practitioners will also recognize that compositions comprising at least two rare sugars as described herein can be added to other items, such as vegetable bodies, including, but are not limited to, mushrooms, celery, peppers, carrots, potatoes, cucumbers, corn, onions, peas, squash and the like.

Also encompassed are food products that include a first food component(s) (e.g. infused food bodies) and a second food component. For example, the food product can be a ready to eat cereals, in which the infused food bodies are an ingredient, along with, e.g., e.g., cereal flakes and the like. Such food products can also be in the form of a mass, e.g., a cereal bar. Infused fruit can be admixed with cereal and formed into a bar such as with a binder. In some embodiments, the bars can include a separate layer or region that includes the infused fruit.

Infused food bodies can also be added to products such as fruit cups, baked goods, confections (e.g., chocolates), and salads (e.g., prepackaged salads and salad kits).

The infused food bodies can be added to a variety of other food products such as dry mixes for baked goods, snack or trail mixes.

The infused food bodies are also suitable for inclusion into a wide variety of dairy products. For example, the infused fruit bodies can be added to yogurt to provide products that provide the nutrition and taste appeal of fruit. Also, the food bodies can be added to a variety of frozen dairy products such as ice cream or soft serve frozen dairy products. The fruit products can be added to nondairy frozen desserts such as sorbets or frozen fruit bars.

Exemplary Consumable Beverage Production Method

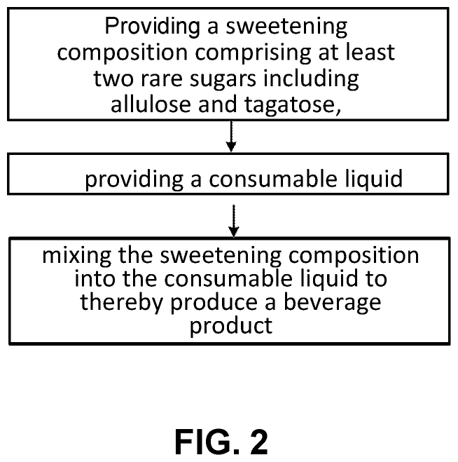

A flow diagram is shown in FIG. 2 of an exemplary combination procedure to provide a consumable beverage in accordance with the present disclosure. In this exemplary procedure, a consumable liquid and at least two rare sugars e.g., allulose and tagatose are provided. The at least two rare sugars are combined (e.g., dissolved, agitated, mixed, blended, and/or stirred) with the consumable liquid to product a consumable beverage product that has an increased sweetness profile. The consumable liquid may be heated while the at least two rare sugars are combined with the consumable liquid to increase the speed of dissolution or increase the saturation of the at least two rare sugars in the consumable liquid. As described in the Examples, the combination of allulose and tagatose was found to produce a synergistic effect on a sweetness profile.

Consumable Beverage Product

The methods described herein can provide a large number of consumable beverage products. A consumable beverage product typically includes a consumable liquid and at least two rare sugars, for example allulose and tagatose. The consumable liquid is infused or combined with at least two rare sugars, e.g., allulose and tagatose, to form a sweetened consumable beverage product.

Generally, the at least two rare sugars may be dissolved in the consumable liquid simultaneously or sequentially in any order. Applicants have determined surprisingly that a combination of at least two rare sugars, e.g., allulose and tagatose, increases the sweetness profile of the composition, food product, or consumable beverage product as compared to using allulose alone or tagatose alone.

Accordingly, in some instances, the consumable beverage product can have, by weight, about 0.1% to about 50% rare sugars, combined, For example, the consumable beverage product can have, by weight, about 0.1% to about 5% rare sugars, e.g., about 1% to about 10% rare sugars, e.g., about 1% to about 20% rare sugars, e.g., about 1% to about 30% rare sugars, e.g., about 1% to about 40% rare sugars, e.g., about 10% to about 20% rare sugars, e.g., about 10% to about 30% rare sugars, e.g., about 10% to about 45% rare sugars, e.g., about 20% to about 25% rare sugars, e.g., about 20% to about 30% rare sugars, e.g., about 30% to about 40% rare sugars, e.g., about 40% to about 50% rare sugars, e.g., about 0.5% to about 1% rare sugars, e.g., about 0.1% to about 2% rare sugars, e.g., about 0.1% to about 3% rare sugars, e.g., about 2% to about 3% rare sugars, e.g., about 3% to about 5% rare sugars, e.g., about 4% to about 6% rare sugars, e.g., about 8% to about 15% rare sugars, e.g., about 15% to about 20% rare sugars, e.g., about 20 to about 40% rare sugars, e.g., or about 1% to about 8%.

A consumable beverage product of the present invention can have, by weight, 0.1% to about 50% allulose. For example, a consumable beverage product can have, by weight, about 0.1% to about 5% allulose, e.g., about 0.1% to about 3.4% allulose, e.g., about 1% to about 10% allulose, e.g., about 1% to about 20% allulose, e.g., about 1% to about 30% allulose, e.g., about 1% to about 40% allulose, e.g., about 10% to about 20% allulose, e.g., about 10% to about 30% allulose, e.g., about 10% to about 45% allulose, e.g., about 20% to about 25% allulose, e.g., about 20% to about 30% allulose, e.g., about 30% to about 40% allulose, e.g., about 40% to about 50% allulose, e.g., about 0.5% to about 1% allulose, e.g., about 0.1% to about 2% allulose, e.g., about 0.1% to about 3% allulose, e.g., about 2% to about 3% allulose, e.g., about 3% to about 5% allulose, e.g., about 4% to about 6% allulose, e.g., about 2% to about 6% allulose, e.g., about 8% to about 15% allulose, e.g., about 15% to about 20% allulose, e.g., about 20 to about 40% allulose, or about 1% to about 8% allulose.

A consumable beverage product of the present invention can have, by weight 0.1% to about 50% tagatose. For example, a consumable beverage product can have, by weight, about 0.1% to about 5% tagatose, e.g., about 0.1% to about 3.4% tagatose, e.g., about 1% to about 10% tagatose, e.g., about 1% to about 20% tagatose, e.g., about 1% to about 30% tagatose, e.g., about 1% to about 40% tagatose, e.g., about 10% to about 20% tagatose, e.g., about 10% to about 30% tagatose, e.g., about 10% to about 45% tagatose, e.g., about 20% to about 25% tagatose, e.g., about 20% to about 30% tagatose, e.g., about 30% to about 40% tagatose, e.g., about 40% to about 50% tagatose, e.g., about 0.5% to about 1% tagatose, e.g., about 0.1% to about 2% tagatose, e.g., about 0.1% to about 3% tagatose, e.g., about 2% to about 3% tagatose, e.g., about 3% to about 5% tagatose, e.g., about 4% to about 6% tagatose, e.g., about 2% to about 6% tagatose, e.g., about 8% to about 15% tagatose, e.g., about 15% to about 20% tagatose, e.g., about 20 to about 40% tagatose, e.g., or about 1% to about 8%.

A consumable beverage product of the present invention can have a ratio of allulose to tagatose, by weight, of about 1:25 to about 25:1. For example, the consumable beverage product can have a ratio of allulose to tagatose, by weight, of about 1:1, e.g., a ratio of allulose to tagatose of about 1:2, e.g., a ratio of allulose to tagatose of about 1:3, e.g., a ratio of allulose to tagatose of about 1:4, e.g., a ratio of allulose to tagatose of about 1:5, e.g., a ratio of allulose to tagatose of about 1:6, e.g., a ratio of allulose to tagatose of about 1:7, e.g., a ratio of allulose to tagatose of about 1:8, e.g., a ratio of allulose to tagatose of about 1:9, e.g., a ratio of allulose to tagatose of about 1:10, e.g., a ratio of allulose to tagatose of about 1:15, e.g., a ratio of allulose to tagatose of about 1:20, e.g., a ratio of allulose to tagatose of about 1:25, e.g., a ratio of allulose to tagatose of about 2:1, e.g., a ratio of allulose to tagatose of about 3:1, e.g., a ratio of allulose to tagatose of about 4:1, e.g., a ratio of allulose to tagatose of about 5:1, e.g., a ratio of allulose to tagatose of about 6:1, e.g., a ratio of allulose to tagatose of about 7:1, e.g., a ratio of allulose to tagatose of about 8:1, e.g., a ratio of allulose to tagatose of about 9:1, e.g., a ratio of allulose to tagatose of about 10:1 e.g., a ratio of allulose to tagatose of about 1:15, e.g., a ratio of allulose to tagatose of about 1:20, or e.g., a ratio of allulose to tagatose of about 1:25.

The at least two rare sugars, e.g., allulose and tagatose, combined with the consumable liquid produce a synergistic phenomenon in which the sweetness profile is increased as compared to using only allulose or only tagatose. Further, the inclusion of both allulose and tagatose in a consumable beverage product with sugar, reduces the amount of sugar needed to create a certain sweetness profile. The addition of the combination of allulose and tagatose maintains the mouthfeel and structure (thinness or thickness) of the consumable beverage product without any added grittiness or noticeable after taste.

In some cases, the consumable beverage product can have a dissolvable solids content of about 12.7° Brix, an acidity of about 0.68%, a pH of about 2.59, about 0.1 to about 50% rare sugar having an allulose to tagatose ratio of about 1:1.

A consumable beverage product of the present invention can have a dissolvable solids content of about 10° Brix to about 15° Brix. For example, a consumable beverage product can have a dissolvable solids content of about 10° Brix to about 15° Brix, e.g., about 10° Brix to about 11° Brix, e.g., about 10° Brix to about 12° Brix, e.g., about 10° Brix to about 13° Brix, e.g., about 10° Brix to about 14° Brix, e.g., about 11° Brix to about 12° Brix, e.g., about 11° Brix to about 13° Brix, e.g., about 11° Brix to about 14° Brix, e.g., about 10° Brix to about 11° Brix, e.g., about 12° Brix to about 13° Brix, e.g., about 12° Brix to about 14° Brix, e.g., about 12° Brix to about 15° Brix, e.g., about 13° Brix to about 14° Brix, e.g., about 13° Brix to about 15° Brix, or e.g., about 14° Brix to about 15° Brix.

A consumable beverage product of the present invention can have an acidity of about 0.5% to about 0.8%. For example a consumable beverage product can have an acidity of about 0.4% to about 0.9%, e.g., about 0.5% to about 0.7%, e.g., about 0.6% to about 0.75%, e.g., about 0.64% to about 0.72%, e.g., about 0.65% to about 0.70%, or e.g., 0.45% to about 0.75%.

A consumable beverage product of the present invention can have a pH of about 2 to about 3.5. For example, a consumable beverage product can have a pH of about 2 to about 3, e.g., about 2.1 to about 3.1, e.g., about 2.2 to about 2.8, e.g., about 2.4 to about 2.7, or about e.g., about 2.5 to about 2.7.

The consumable liquid may be e.g., a water based beverage, fruit juice, fruit juice cocktail (e.g., cranberry juice cocktail), fruit concentrate, fruit extract, vegetable juice, vegetable concentrate, vegetable extract, lemonade, sports drink, vegetable stock, chicken stock, beef stock, a soft drink (soda), a flavoring syrup, carbonated beverage, seltzer, hard seltzer, sweetening syrup (e.g., maple syrup, agave, honey, liquid artificial sweetener, simple syrup), an alcoholic beverage, cocktail mix, smoothie, dairy based liquid, shake, dairy-alternative beverage, tea, or coffee.

In some instances, the consumable liquid may be a fruit or vegetable juice. A fruit or vegetable juice is typically a juice derived from a fruit or vegetable with a water content, by weight, of about 50% to about 99% (e.g., about 80% to about 90%). The fruit juice may be a berry juice, (e.g., cranberry juice, blueberry juice, strawberry juice, raspberry juice, blackberry juice, or elderberry juice), orange juice, peach juice, grape juice, apple juice, grapefruit juice, watermelon juice, kiwi juice, mango juice, cherry juice, lime juice, lemon juice, pear juice, pineapple juice, pomegranate juice, papaya juice or any combination thereof. Vegetable juice may be cucumber juice, pepper juice, beet juice, celery juice, wheatgrass juice, carrot juice, spinach juice, kale juice, broccoli juice, parsley juice, chard juice, sweet potato juice, or any combination thereof.

In some instances, the consumable liquid may be a fruit or vegetable concentrate. A fruit or vegetable concentrate is typically a juice derived from a fruit or vegetable that has been dehydrated to a water content, by weight, of about 20% to about 50%. The fruit concentrate may be a berry concentrate, (e.g., cranberry concentrate, blueberry concentrate, strawberry concentrate, raspberry concentrate, blackberry concentrate, or elderberry concentrate), orange concentrate, peach concentrate, grape concentrate, apple concentrate, grapefruit concentrate, pear concentrate, watermelon concentrate, kiwi concentrate, tomato juice, mango concentrate, cherry concentrate, lime concentrate, lemon concentrate, pineapple concentrate, pomegranate concentrate, papaya concentrate, or any combination thereof. The vegetable concentrate may be cucumber concentrate, pepper concentrate, beet concentrate, celery concentrate, wheatgrass concentrate, carrot concentrate, spinach concentrate, kale concentrate, broccoli concentrate, parsley concentrate, chard concentrate, sweet potato concentrate, or any combination thereof.

In some instances, the consumable liquid may be a fruit or vegetable extract. A fruit or vegetable extract is typically a juice derived from a fruit or vegetable that has been dehydrated to a water content, by weight, of about 0% to about 20% (e.g., about 5% to about 18%). The fruit extract may be a berry extract, (e.g., cranberry extract, blueberry extract, strawberry extract, raspberry extract, blackberry extract, or elderberry extract), orange extract, peach extract, grape extract, apple extract, grapefruit extract, watermelon extract, kiwi extract, pear extract, mango extract, cherry extract, lime extract, lemon extract, pineapple extract, pomegranate extract, or papaya extract. The vegetable extract may be cucumber extract, pepper extract, beet extract, celery extract, wheatgrass extract, carrot extract, spinach extract, kale extract, broccoli extract, parsley extract, chard extract, sweet potato extract, or any combination thereof.

A consumable beverage product may include, by weight, about 0% to about 60% sucrose (e.g., about 4% to about 10%). The ratio of sucrose to the at least two rare sugars can be about 1 (i.e., about equal sucrose to rare sugars), greater than one, (more sucrose than rare sugars), or less than one (more rare sugars than sucrose). The sucrose may be naturally derived sucrose present in the consumable liquid, for example naturally occurring sugars in fruits or added to the consumable liquid. A consumable beverage product can have a water content, by weight, of about 0% to about 99%, e.g., about 10% to about 80%, e.g., about 20% to about 60%, e.g., about 40% to about 70%, e.g., about 25% to about 50%, e.g., about 50% to about 75%, e.g., about 20% to about 30%, e.g., about 20% to about 50%, e.g., about 40% to about 80%, e.g., about 50% to about 80%, e.g., about 80% to about 90%, or e.g., about 45% to about 65%. The consumable beverage product may further include ascorbic acid, sugar, vegetable concentrate, and/or cranberry concentrate.

### Beverage Product Examples

Beverage Product A

| | | |
|---|---|---|
| 1. | Water | 85.425% w/w |
| 2. | Sucrose-Domestic Dry Sugar | 6.939% w/w |
| 3. | Cranberry Concentrate Type C 50° Brix Bulk | 2.824% w/w |
| 4. | Cranberry Concentrate-Essence Returned | 1.211% w/w |
| 5. | Ascorbic Acid | 0.079% w/w |
| 6. | GNT Juneberry (natural color) | 0.023% w/w |
| 7. | Crytsalline Allulose | 1.750% w/w |
| 8. | Crystalline Tagatose | 1.750% w/w |

Sweetener Compositions

The present invention further relates to a sweetener composition formulated to increase the sweetness profile of a food or beverage product. A sweetener composition typically includes at least two rare sugars, for example allulose and tagatose. In some cases, the sweetener composition also includes bulking agent. The bulking agent may be soluble corn fiber, soluble tapioca fiber, malto-oligosaccharides, fructo-oligosaccharides, sugar. The sweetener composition can also include high intensity sweeteners (e.g., sucralose, stevia, or other natural flavors). The sweetener composition may be in any form, e.g., in liquid, solid (e.g., cube, tablet, pill, or the like), or powder form. The sweetener composition may be used in the preparation of food or beverages, for example as a full or partial sugar replacement in food and/or beverage preparation. In some cases, the rare sugar, e.g., allulose and/or tagatose are the bulking agent(s) or part of the bulking agents.

A sweetener composition, in some instances, can have, by weight, about 0.1% to about 100% rare sugars, combined. For example, the sweetener composition can have, by weight, about 0.1% to about 5% rare sugars, e.g., about 1% to about 10% rare sugars, e.g., about 1% to about 20% rare sugars, e.g., about 1% to about 30% rare sugars, e.g., about 1% to about 40% rare sugars, e.g., about 10% to about 20% rare sugars, e.g., about 10% to about 30% rare sugars, e.g., about 10% to about 45% rare sugars, e.g., about 20% to about 25% rare sugars, e.g., about 20% to about 30% rare sugars, e.g., about 30% to about 40% rare sugars, e.g., about 40% to about 50% rare sugars, e.g., about 0.5% to about 1% rare sugars, e.g., about 0.1% to about 2% rare sugars, e.g., about 0.1% to about 3% rare sugars, e.g., about 2% to about 3% rare sugars, e.g., about 3% to about 5% rare sugars, e.g., about 4% to about 6% rare sugars, e.g., about 8% to about 15% rare sugars, e.g., about 15% to about 20% rare sugars, e.g., about 20% to about 40% rare sugars, e.g., about 1% to about 8%, e.g., about 60% to about 70% rare sugars, e.g., about 70% to about 80% rare sugars, e.g., about 80% to about 90% rare sugars, e.g., about 90% to about 99% rare sugars, e.g., about 50% to about 75% rare sugars, e.g., about 75% to about 99% rare sugars, e.g., about 60% to about 80% rare sugars, e.g., about 30% to about 60% rare sugars, e.g., about 25 to about 75% rare sugars, or e.g., about 15 to about 60% rare sugars.

A sweetener composition of the present invention can have, by weight, about 0.1% to about 75% allulose. For example, the sweetener composition can have, by weight, about 0.1% to about 5% allulose, e.g., about 0.1% to about 3.4% allulose, e.g., about 1% to about 10% allulose, e.g., about 1% to about 20% allulose, e.g., about 1% to about 30% allulose, e.g., about 1% to about 40% allulose, e.g., about 10% to about 20% allulose, e.g., about 10% to about 30% allulose, e.g., about 10% to about 45% allulose, e.g., about 20% to about 25% allulose, e.g., about 20% to about 30% allulose, e.g., about 30% to about 40% allulose, e.g., about 40% to about 50% allulose, e.g., about 0.5% to about 1% allulose, e.g., about 0.1% to about 2% allulose, e.g., about 0.1% to about 3% allulose, e.g., about 2% to about 3% allulose, e.g., about 3% to about 5% allulose, e.g., about 4% to about 6% allulose, e.g., about 2% to about 6% allulose, e.g., about 8% to about 15% allulose, e.g., about 15% to about 20% allulose, e.g., about 20% to about 40% allulose, e.g., about 1% to about 8%, e.g., about 25% to about 75% allulose, e.g., about 50% to about 75% allulose, e.g., about 50% to about 60% allulose, e.g., about 60% to about 70% allulose, e.g., about 15% to about 60% allulose, e.g., about 30% to about 55% allulose, or e.g., about 45% to about 75% allulose.

A sweetener composition of the present invention can have, by weight 0.1% to about 75% tagatose. For example, the sweetener composition can have, by weight, about 0.1% to about 5% tagatose, e.g., about 0.1% to about 3.4% tagatose, e.g., about 1% to about 10% tagatose, e.g., about 1% to about 20% tagatose, e.g., about 1% to about 30% tagatose, e.g., about 1% to about 40% tagatose, e.g., about 10% to about 20% tagatose, e.g., about 10% to about 30% tagatose, e.g., about 10% to about 45% tagatose, e.g., about 20% to about 25% tagatose, e.g., about 20% to about 30% tagatose, e.g., about 30% to about 40% tagatose, e.g., about 40% to about 50% tagatose, e.g., about 0.5% to about 1% tagatose, e.g., about 0.1% to about 2% tagatose, e.g., about 0.1% to about 3% tagatose, e.g., about 2% to about 3% tagatose, e.g., about 3% to about 5% tagatose, e.g., about 4% to about 6% tagatose, e.g., about 2% to about 6% tagatose, e.g., about 8% to about 15% tagatose, e.g., about 15% to about 20% tagatose, e.g., about 20% to about 40% tagatose, e.g., about 1% to about 8%, e.g., about 25% to about 75% tagatose, e.g., about 50% to about 75% tagatose, e.g., about 50% to about 60% tagatose, e.g., about 60% to about 70% tagatose, e.g., about 15% to about 60% tagatose, e.g., about 30% to about 55% tagatose, or e.g., about 45% to about 75% tagatose.

A sweetener composition of the present invention can have a ratio of allulose to tagatose, by weight, of about 1:25 to about 25:1. For example, a sweetener composition can have a ratio of allulose to tagatose, by weight, of about 1:1, e.g., a ratio of allulose to tagatose of about 1:2, e.g., a ratio of allulose to tagatose of about 1:3, e.g., a ratio of allulose to tagatose of about 1:4, e.g., a ratio of allulose to tagatose of about 1:5, e.g., a ratio of allulose to tagatose of about 1:6, e.g., a ratio of allulose to tagatose of about 1:7, e.g., a ratio of allulose to tagatose of about 1:8, e.g., a ratio of allulose to tagatose of about 1:9, e.g., a ratio of allulose to tagatose of about 1:10, e.g., a ratio of allulose to tagatose of about 1:15, e.g., a ratio of allulose to tagatose of about 1:20, e.g., a ratio of allulose to tagatose of about 1:25, e.g., a ratio of allulose to tagatose of about 2:1, e.g., a ratio of allulose to tagatose of about 3:1, e.g., a ratio of allulose to tagatose of about 4:1, e.g., a ratio of allulose to tagatose of about 5:1, e.g., a ratio of allulose to tagatose of about 6:1, e.g., a ratio of allulose to tagatose of about 7:1, e.g., a ratio of allulose to tagatose of about 8:1, e.g., a ratio of allulose to tagatose of about 9:1, e.g., a ratio of allulose to tagatose of about 10:1 e.g., a ratio of allulose to tagatose of about 1:15, e.g., a ratio of allulose to tagatose of about 1:20, or e.g., a ratio of allulose to tagatose of about 1:25.

A sweetener composition may include, by weight, about 0% to about 60% sucrose. The ratio of sucrose to the at least two rare sugars can be about 1 (about equal sucrose to rare sugars), greater than one, (more sucrose than rare sugars), or less than one (more rare sugars than sucrose). The sucrose may be naturally derived sucrose present the consumable liquid, for example naturally occurring sugars in fruits or added to the consumable liquid. the sweetener composition can have a water content, by weight, of about 0% to about 99%, e.g., about 10% to about 80%, e.g., about 20% to about 60%, e.g., about 40% to about 70%, e.g., about 25% to about 50%, e.g., about 50% to about 75%, e.g., 20% to about 30%, e.g., 20% to about 50%, e.g., about 40% to about 80%, e.g., about 50% to about 80%, or e.g., about 45% to about 65%.

The sweetener composition may further include low-caloric sweeteners, for example artificial sweeteners and/or sugar alcohols, at about 0% to about 60%. The artificial sweeteners can be a combination of known artificial sweeteners or a single artificial sweetener. Exemplary artificial sweetener include, e.g., Saccharin, Aspartame, Acesulfame Potassium, Sucralose, Advantame, and/or Neotame. Some sweetener compositions can include sugar alcohols (e.g., Erythritol, Isomalt, Lactitol, Maltitol, Mannitol, Sorbitol, Xylitol) at about 0% to about 60%.

The sweetener composition may be packaged in predetermined weights as sugar alternative packets, (e.g., for adding to coffee or tea) or in larger weights as sugar alternatives (e.g., for baking or cooking). For example, the sweetener composition can have a weight, about 0.1 g to about 10 g, e.g., about 0.25 g and about 5 g, e.g., about 0.2 g to about 0.75 g, e.g., about 0.25 g to about 1 g, e.g., about 1 g to about 3 g, e.g., about 0.5 g to about 2 g, e.g., about 0.5 g to about 3 g, e.g., about 1 g to about 4 g, e.g., about 1 g to about 10 g, e.g., about 2 g to about 6 g, e.g., about 3 g to about 9 g, e.g., about 6 g to about 10 g, e.g., about 7 g to about 9 g, e.g., 5 g to about 7 g, or e.g., about 5 g to about 8 g. In some instances, the sweetener composition can have a weight between 10 g and 5 kg, e.g., about 10 g to about 20 g, e.g., about 50 g to about 100 g, e.g., about 100 g to about 200 g, e.g., about 200 g to about 300 g, e.g., about 300 g to about 400 g, e.g., about 400 g to about 500 g, e.g., about 500 g to about 600 g, e.g., about 600 g to about 700 g, e.g., about 700 g to about 800 g, e.g., about 800 g to about 90 g, e.g., about 900 g to about 1 kg, e.g., about 1 kg to about 2 kg, e.g., about 2 kg to about 3 kg, e.g., about 3 kg to about 4 kg, e.g., about 4 kg to about 5 kg, e.g., about 25 g to about 75 g, e.g., about 150 g to about 300 g, e.g., about 350 g to about 400 g, e.g., about 450 g to about 600 g, or e.g., about 100 g to about 800 g.

### Examples

Experiment 1

Cranberry hulls (fruit bodies) were each infused with an infusion formulation under the same conditions using a counter current apparatus. The countercurrent apparatus flows the infusion formulation at a temperature of 110° F. The infusion formulation is provided at a 3:1 ratio, by weight, of infusion formulation to cranberry hulls (fruit bodies). The cranberry hulls may have previously undergone extraction to produce extracted cranberry hulls. The countercurrent infusion runs for about 60 to about 75 minutes. Cranberry 1 was infused with an infusion formulation that included 22% allulose. The infusion component of Cranberry 1 therefore has an allulose content of 22%. Cranberry 2 was infused with an infusion formulation that included 22% tagatose. The infusion component of Cranberry 2 therefore has a tagatose content of 22%. Cranberry 3 was infused with an infusion formulation that included 11% allulose and 11% tagatose. The infusion component of Cranberry 3 therefore has an allulose content of 11% and a tagatose content of 11%. The infused cranberries were dried after infusion, producing sweetened dried cranberries.

The physical properties (e.g., Brix, Acidity, % Moisture, Water activity, Piece count/100 grams, Texture analysis and Color) were measured for Cranberry 1, 2, and 3, shown in Table 1 below.

TABLE 1

| | Cranberry | Cranberry | Cranberry 3: |
|---|---|---|---|
| | 1: Only | 2: Only | Allulose and |
| | Allulose | Tagatose | Tagatose |
| Variable | Infused | Infused = | Infused |
| Brix | 76 | 78 | 76.3 |
| Acidity % | 1.80 | 1.71 | 1.72 |
| % Moisture | 18 | 17 | 18 |
| Water Activity | 0.590 | 0.60 | 0.585 |
| Piece Ct/100 g | 78 | 83 | 70 |
| Less pieces = | | | |
| more plump | | | |
| Texture | 6007 | 6078 | 7284 |
| analysis- | | | |
| Peak Positive | | | |
| Force | | | |
| (higher | | | |
| number = | | | |
| firmer texture) | | | |
| Color chart | 3 | 3 | 3 |
| (1-5) | | | |
| 1 = lightest red | | | |
| 5 = darkest red | | | |

Cranberry 1, cranberry 2, and cranberry 3, were also evaluated by a panel of six sensory experts. The experts evaluated color, texture, aroma, appearance (plumpness) sweetness (range 1-10, low to high sweetness—with reference 7), bitterness or any lingering aftertaste, sour, overall flavor, and overall liking. The results of the sensory panel are shown below in Table 2.

| | | | Allulose and |
|---|---|---|---|
| | Allulose | Tagatose | Tagatose |
| | infused | Infused | Infused together |
| Sweetness | Low | Low | Higher |
| (range 1-10, | sweetness- | sweetness- | sweetness- |
| low to high | Score = 3 | Score 3.5 | Score 4.75 |
| sweetness- | | | |
| with | | | |
| reference 7), | | | |
| Bitterness | Mid-level | Slight | Low |
| or any | bitterness- | bitterness | bitterness |
| Lingering | lingers | at end | |
| After | | | |
| Taste | | | |
| Sourness | Mid-level | High-level | Low-level |
| | sour | sour | sour |
| Aroma | Low aroma | Low aroma | Small amount |
| | | | of aroma |
| Color | Good color | Good color | Good color |
| Texture | Good | Slightly | Good |
| | texture- | chewy | texture- |
| | soft | texture, | softest of |
| | | less soft | three |
| Appearance | Good | Mid- | Good |
| (Plumpness) | plumpness/ | plumpness/ | plumpness/ |
| | appearance | appearance | appearance |
| Flavor and | Fair overall | Fair overall | Good overall |
| Overall | flavor and | flavor and | flavor and |
| Liking | liking | liking | liking |

As is clear from the results of the sensory panel, the combination of allulose and tagatose together has a greater sweetness level, a fruitiest flavor, a more balanced tart/sweet profile, a stronger upfront sweetness and flavor intensity that carried through.

Experiment 2

A consumable beverage product (Juice 1) was produced by dissolving allulose and tagatose in a consumable liquid (25% Reduced Sugar Cranberry Juice Cocktail). The consumable beverage product was evaluated by an expert sensory panel against a Control Juice, Juice 2, and Juice 3. The control juice is a Full Sugar Cranberry Cocktail. Juice 2 is a 25% Reduced Sugar Cranberry Cocktail with 3.5% Allulose. Juice 3 is a 25% Reduced sugar Cranberry Cocktail with 3.5% Tagatose.

The physical properties (e.g., ° Brix, Titrate Acidity, and pH) were measured for Control Juice, Juice 1, Juice 2, and Juice 3, shown in Table 3 below.

TABLE 3

| | | Juice 1 | | |
|---|---|---|---|---|
| | Control | (Allulose | | |
| | Juice | 1.75%, | Juice 2 | Juice 3 |
| | (Full | Tagatose | Allulose | (Tagatose |
| | Sugar) | 1.75%) | (3.5%) | 3.5%) |
| Brix | 11.40 | 12.71 | 12.85 | 12.72 |
| Acidity % | 0.67% | 0.68% | 0.69% | 0.68% |
| (w/v) | | | | |
| pH | 2.62 | 2.59 | 2.61 | 2.62 |

The Control Juice and Juices 1, 2, and 3 were also blindly evaluated by a panel of six sensory experts in comparison the Full Sugar Cranberry Juice Cocktail. The experts evaluated aroma, flavor, sweetness, and overall experience. The results of the sensory panel are shown below in Table 4. Each evaluated feature is ranked 0 to 5, 0 being no difference in comparison to the Full sugar Cranberry Juice Cocktail and 5 being a large difference between the tested juice and the full sugar cranberry cocktail.

TABLE 4

| | | Juice 1 | | |
|---|---|---|---|---|
| | Blind | (Allulose | | |
| | Juice | 1.75%, | Juice 2 | Juice 3 |
| | (Full | Tagatose | Allulose | (Tagatose |
| | Sugar) | 1.75%) | (3.5%) | 3.5%) |
| Aroma | 0/5 | 0/5 | 1/5 | 0/5 |
| | no | no | Extremely | no |
| | difference | difference | close | difference |
| | | | can't tell | |
| Flavor | 2/5 | 2.5/5 | 2/5 | 2/5 |
| | Close but | Close but | Close but | Close |
| | you can | you can tell a | you can | but you |
| | tell a | difference/ | tell a | can tell a |
| | difference | different | difference | difference |
| Sweetness | 1.5/5 | 2.5/5 | 1/5 | 1/5 |
| | No | Close but | Extremely | Extremely |
| | difference/ | you can tell a | close | close can't |
| | Extremely | difference/ | can't tell a | tell a |
| | close can't | different | difference | difference |
| | tell a | | | |
| | difference | | | |
| Overall | 1/5 | 2.5/5 | 2/5 | 2/5 |
| | Extremely | Close but | Close but | Close |
| | close | you can tell/ | you can | but you |
| | can't tell a | different | tell | can tell |
| | difference | | | |
| Comments | “blind?” | “tasted sweeter | “lacking | “more |
| | “very similar | than control” | cranberry | caramelized |
| | to ref, | “blind | flavor and | notes |
| | maybe less | control?” | astringency” | than ref” |
| | sweet?” | “Almost more | “lacks | “less sweet, |
| | “This is | sweet than | cranberry | less |
| | pretty close- | ref, lost | flavor | roundness, |
| | feels like the | some tartness | intensity” | more |
| | mouthfeel | and cranberry | “slight | bitterness at |
| | is slightly | flavor” | papery | end” |
| | thinner” | “very slightly | aroma, | “sweetness |
| | “doesn't | higher | sweetness at | is pretty |
| | taste as | fermented | end is more | similar |
| | juicy or | aroma. This | intense, | but overall |
| | fresh, flavor | was my | caramelized | flavor is |
| | profile is | last sample, | notes” | more of |
| | similar.” | which might | | a generic |
| | | be why it | | red berry |
| | | seems less | | than |
| | | flavorful.” | | cranberry. |
| | | | | Very |
| | | | | slightly |
| | | | | more |
| | | | | fermented |
| | | | | aroma” |

As is clear from the results of the sensor panel, the experts noticed a sweeter profile in the juice that uses both allulose and tagatose (Juice 1). Juice 1 was also shown to have a greater difference in flavor from the full sugar juice as compared to Juices 2 and 3.

### OTHER EMBODIMENTS

While many of the infusion formulations and products described herein include at least two rare sugars, skilled practitioners will appreciate that in some instances a single rare sugar can be used. For example, some infusion formulations described include a single rare sugar (e.g., allulose). For example, the infusion formulation can have, by weight, 0.1% to about 50% allulose. For example, an infusion formulation can have about 0.1% to about 5% allulose, e.g., about 0.1% to about 3.4% allulose, e.g., about 1% to about 10% allulose, e.g., about 1% to about 20% allulose, e.g., about 1% to about 30% allulose, e.g., about 1% to about 40% allulose, e.g., about 10% to about 20% allulose, e.g., about 10% to about 30% allulose, e.g., about 10% to about 45% allulose, e.g., about 20% to about 25% allulose, e.g., about 20% to about 30% allulose, e.g., about 30% to about 40% allulose, e.g., about 40% to about 50% allulose, e.g., about 0.5% to about 1% allulose, e.g., about 0.1% to about 2% allulose, e.g., about 0.1% to about 3% allulose, e.g., about 2% to about 3% allulose, e.g., about 3% to about 5% allulose, e.g., about 4% to about 6% allulose, e.g., about 2% to about 6% allulose, e.g., about 8% to about 15% allulose, e.g., about 15% to about 20% allulose, e.g., about 20 to about 40% allulose, e.g., about 35 to about 40% allulose, e.g., about 20 to about 45% allulose, e.g., about 35% to about 45% allulose, or about 1% to about 8% allulose.

The infusion formulation can have a Brix level of 40° Brix to about 70° Brix, e.g., for example about 60° Brix to about 70° Brix, about 64° Brix, about 60° Brix, about 61° Brix, about 62° Brix, about 62° Brix, about 65° Brix, about 66° Brix, about 67° Brix, about 68° Brix, about 69° Brix or about 70° Brix.

General exemplary infusion formulations are described below, which may be used in any of the methods described herein and do not limit the scope of the invention described in the claims. All percentage values are provided on a weight/weight basis:

Formulation B Infusion Syrup

| | | |
|---|---|---|
| 1. | Allulose | 26.6% w/w |
| 2. | Glycerin | 9.6% w/w |
| 3. | Soluble Corn Fiber | 46.8% w/w |
| 4. | Cranberry Concentrate | 8.0% w/w |
| 5. | Water | 8.6% w/w |
| 6. | Natural Sweetness Modifier | 0.32% w/w |

Formulation C Infusion Syrup

| | | |
|---|---|---|
| 1. | Allulose | 20-40% w/w |
| 2. | Glycerin | 5-15% w/w |
| 3. | Soluble Corn Fiber | 40-60% w/w |
| 4. | Cranberry Concentrate | 5-15% w/w |
| 5. | Water | 5-15% w/w |
| 6. | Natural Sweetness Modifier | 0.20-0.40% |
| | | w/w |

While an infused product typically including a food component (i.e., the natural and/or endogenous material of the infused food product, e.g., a fruit hull, such as a cranberry or blueberry hull) and an infusion component (i.e., one or more exogenously-added infusion formulation(s) described herein) that comprises at least two rare sugars has been described, some infused products have an infusion components that comprises a single rare sugar (e.g., allulose). In some instances, the infused product will include a fruit (e.g., cranberry or blueberry) hull that comprises within it one or more infusion formulation(s), wherein the infusion formulation(s) occupies (e.g., partially or completely) one or more voids left within the fruit hull as a result of the fruit being treated in an extraction process (e.g., squeezing, subcritical water extraction, and/or countercurrent extraction) prior to being treated with the infusion process.

For example, any type of fruit body that can be infused with an infusion formulation and retain a substantial amount of the infusion formulation within the body (i.e., retain infusion formulation at least about 0.1%, e.g., at least about 1%, 2%, 5%, 8%, 10%, 20% or at least about 30% total weight of the food body after infusion) after removal of the food body from the infusion formulation, can be used and is included in the invention in its infused form. The infused food bodies can be, e.g., infused dried fruit, for example sweetened dried cranberries. In some embodiments, the infused food bodies, e.g., infused cranberries, will have infused solids content of at least or about 40° Brix, e.g., at least or about 50, 55, 60, 65, 70, or 75° Brix, before drying of the food body.

In some instances, the infused food products may be characterized by including a rare sugar (e.g., allulose) content of at least or about 1% by weight. For example, the infused food bodies can have a rare sugar content ranging from about 1% to about 65%, e.g., about 1% to about 5%, e.g., about 2% to about 5%, e.g., about 3% to about 5%, e.g., about 1% to about 3%, e.g., about 0.5% to about 2.5%, e.g., about 0.5% to about 3.5%, e.g., about 2% to about 5%, e.g., 1% to about 10, e.g., about 1% to about 8%, e.g., about 1% to about 7%, e.g., about 5% to about 50%, e.g., about 35% to about 50%, e.g., about 45% to about 50%, e.g., about 25% to about 45%, e.g., about 35% to about 45%, e.g., about 30% to about 40%, e.g., about 25% to about 35%, e.g., about 20% to about 50%, or e.g., about 10% to about 20%, by weight. In some embodiments, the infused food bodies can have a rare sugars content (e.g., allulose content) of about 1.5% to about 3.5%, e.g., about 2.0% to about 2.5%, e.g., a content of at least or about 1% by weight.

In certain embodiments, the food products include food bodies that are infused with a rare sugar, e.g., for example allulose. In some cases, the infusion formulation includes a rare sugar and sucrose. In some instances, the infusion formulation may comprise from about least about 0.1%, e.g., at least about 0.5%, e.g., at least about 1%, e.g., at least about 2%, e.g., at least about 3%, e.g., at least about 4%, e.g., at least about 5%, e.g., at least about 6%, e.g., at least about 7%, e.g., at least about 8%, e.g., at least about 9%, e.g., at least about 10%, e.g., at least about 11%, e.g., at least about 12%, e.g., at least about 13%, e.g., at least about 14%, e.g., at least about 15%, e.g., at least about 16%, e.g., at least about 17%, e.g., at least about 18%, e.g., at least about 19%, e.g., at least about 20%, e.g., at least about 21%, e.g., at least about 22%, e.g., at least about 23%, e.g., at least about 24%, e.g., at least about 25%, e.g., at least about 30%, e.g., at least about 40%, e.g., at least about 50%, e.g., at least about 60%, e.g., at least about 70%, e.g., at least about 80%, e.g., at least about 90 or e.g., at least about 98%, of the total weight of the infused food body. The infused food bodies can be in dried, semi-dried, or non-dried form. The infusion component and/or the food components can further include sucrose, for example about 0% to about 60% sucrose.

In one example the food product, e.g., an infused cranberry, can have an acidity of about 1.80%, a Brix level of at least 77° Brix, and a water activity of about 0.53. In some cases, the food product, e.g., an infused fruit body can have an acidity of about 1.60% to about 2.00%, a Brix level of about 70° Brix to about 95° Brix, and a water activity of about 0.48 to about 0.58.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A product, comprising:
a food body comprising:
a food component; and
an infusion component, wherein the infusion component comprises at least two rare sugars including tagatose and allulose.

2. The product of claim 1, wherein the food body is a fruit body.

3. The product of claim 2, wherein the fruit body is a fruit selected from the group consisting of cranberries, blueberries, cherries, grapes, mangos, pineapples, raspberries, blackberries, dates, apples, apricots, lingonberries, tomatoes, huckleberries, chokeberries, figs, gooseberries, elderberries, strawberries, plums, pears, and peaches.

4. The product of claim 3, wherein the fruit body is a cranberry.

5. The product of claim 1, wherein the infusion component comprises tagatose and allulose in a ratio of about 1:1, about 2:1, about 3:1, about 4:1, about 5:1, about 1:2, about 1:3, about 1:4, or about 1:5.

6-7. (canceled)

8. The product of claim 1, wherein the product has a Brix level of about 70-80° Brix.

9-11. (canceled)

12. The product of claim 1, wherein the product has an acidity of about 1.72%, a moisture level of about 18, and a water activity of about 0.585.

13. The product of claim 1, wherein the product has a peak positive force of about 6000 grams per second (g/sec) to about 8000 (g/sec).

14-16. (canceled)

17. The product of claim 1, wherein the food body is an infused food body having an infused volume, wherein the infused volume is less than a volume of the food body prior to infusion.

18-19. (canceled)

20. The product of claim 1, wherein the product has a moisture content of about 15% to about 20%.

21-22. (canceled)

23. The product of claim 1, wherein the product has a water activity of about 0.4 to about 0.7.

24-25. (canceled)

26. The product of claim 1, wherein the product has an acidity of about 1% to about 2%.

27-36. (canceled)

37. A method for infusing a food product, the method comprising:
providing a food body,
providing an infusion formulation having at least two rare sugars including tagatose and allulose,
infusing the food body with the infusion formulation, and
collecting the food body, to thereby a produce an infused food body.

38. (canceled)

39. The method of claim 37, further comprising, prior to infusing the food body with the infusion formulation, extracting juice from the food body.

40. (canceled)

41. The method of claim 37, wherein the infusion formulation has a viscosity of about 100 to about 2000 cps.

42. The method of claim 37, wherein the infusion formulation has a dissolved solids content of about 40° Brix to about 70° Brix.

43. (canceled)

44. The method of claim 37, wherein the food body is a fruit body or vegetable body.

45-145. (canceled)

146. A food product, comprising:
a food body comprising:
a food component; and
an infusion component, wherein the infusion component comprises an allulose content of about 5% to about 50%.

147. The food product of claim 146, wherein the food body is a fruit body.

148. (canceled)

149. (canceled)

150. The food product of claim 149, wherein the cranberry has an acidity of about 1.80% and a water activity of about 0.53 and a Brix level of at least 77° Brix.

151-152. (canceled)

153. An infusion formulation comprising by weight/weight:
about 5% to about 15% water;
about 5% to about 15% cranberry juice concentrate;
about 40% to about 60% soluble corn fiber; and
about 20% to about 40% allulose.

154. The infusion formulation of claim 153, further comprising about 5% to about 15% glycerin, by weight.

155. The infusion formulation of claim 153, wherein the infusion formulation has a Brix level of about 60° Brix to about 70° Brix.

156. The infusion formulation of claim 153, further comprising about 0.20% to about 0.40% natural sweetness modifier, by weight.
